(19) United States
(12) Patent Application Publication
(10) Pub. No.: US 2002/0106672 A1
(43) Pub. Date: Aug. 8, 2002

(54) Nucleotide sequences coding for the hisC2 gene

(75) Inventors: Mike Farwick, Bielefeld (DE); Klaus Huthmacher, Gelnhausen (DE); Brigitte Bathe, Salzkotten (DE); Walter Pfefferle, Halle (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Degussa AG, Hanau (DE)

(21) Appl. No.: 09/948,649

(22) Filed: Sep. 10, 2001

(30) Foreign Application Priority Data
Sep. 9, 2000 (DE) 100 44 709.0
Feb. 23, 2001 (DE) 101 08 838.8

(51) Int. Cl. C12Q 1/68; C07H 21/04; C12N 9/10; C12P 21/02; C12N 1/21

(52) U.S. Cl. 435/6; 435/69.1; 435/252.3; 435/193; 536/23.2; 435/320.1

(57) ABSTRACT

The invention relates to polynucleotides corresponding to the hisC2 gene and which encode a histidinol phosphate aminotransferase, methods of producing L-amino acids, and methods of screening for polynucleotides which encode proteins having histidinol phosphate aminotransferase activity.

### CROSS-REFERENCE TO RELATED APPLICATIONS

The present application claims priority to German Application No. DE 10108838.8 filed Feb. 23, 2001 and German Application No. DE 10044709.0 filed Sep. 09, 2000; the entire contents of both applications are incorporated herein by reference.

### BACKGROUND OF THE INVENTION

1. Field Of the Invention

The invention provides nucleotide sequences from Coryneform bacteria coding for the hisC2 gene and a process for the fermentative preparation of amino acids using bacteria in which the hisC2 gene is attenuated. The hisC2 gene codes for histidinol phosphate aminotransferase.

2. Discussion Of the Background

L-amino acids, particularly L-lysine, are used in human medicine, in the pharmaceutical industry, in the food industry, and, in particular in animal nutrition.

It is known that amino acids are prepared by fermentation from strains of Coryneform bacteria, particularly Corynebacterium glutamicum. Due to its great importance, attempts are constantly being made to improve the preparation process. Improvements to the process may concern measures relating to fermentation for example, agitation and oxygen supply, or the composition of the nutrient media, such as the sugar concentration during fermentation, or isolating the product form by, for example, ion exchange chromatography or the intrinsic output properties of the microorganism itself.

The output properties of these microorganisms are improved by employing methods of mutagenesis, selection, and mutant selection. These methods yield strains that produce amino acids and are resistant to antimetabolites or are auxotrophic for metabolites of regulatory importance.

For a number of years, methods of recombinant DNA technology have also been used to improve L-amino acid producing strains of Coryneform bacteria by amplifying individual amino acid biosynthesis genes and examining the effect on amino acid production. However, there remains a critical need for improved methods of producing L-amino acids and thus for the provision of strains of bacteria producing higher amounts of L-amino acids. On a commercial or industrial scale even small improvements in the yield of L-amino acids, or the efficiency of their production, are economically significant. Prior to the present invention, it was not recognized that attenuated expression of the hisC2 gene would improve L-amino acid yields.

### SUMMARY OF THE INVENTION

An object of the present invention is to provide novel measures for the improved production of L-amino acids or amino acids where these amino acids include L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine, and in particular L-lysine and the salts (monohydrochloride or sulfate) thereof.

One object of the present invention is providing a novel process for improving the fermentative production of said L-amino acids, particularly L-lysine. Such a process includes enhanced bacteria, preferably enhanced Coryneform bacteria, which express attenuated amounts of the histidinol phosphate aminotransferase, which is encoded by the hisC2 gene.

Thus, another object of the present invention is providing such a bacterium, which expresses an attenuated amount of histidinol phosphate aminotransferase or gene products of the hisC2 gene.

Another object of the present invention is providing a bacterium, preferably a Coryneform bacterium, which expresses a polypeptide that has attenuated histidinol phosphate aminotransferase activity.

Another object of the invention is to provide a nucleotide sequence encoding a polypeptide which has histidinol phosphate aminotransferase protein sequence. One embodiment of such a sequence is the nucleotide sequence of SEQ ID NO: 1.

A further object of the invention is a method of making histidinol phosphate aminotransferase or an isolated polypeptide having histidinol phosphate aminotransferase activity, as well as use of such isolated polypeptides in the production of amino acids. One embodiment of such a polypeptide is the polypeptide having the amino acid sequence of SEQ ID NO: 2.

Other objects of the invention include methods of detecting nucleic acid sequences homologous to SEQ ID NO: 1, particularly nucleic acid sequences encoding polypeptides that have histidinol phosphate aminotransferase activity, and methods of making nucleic acids encoding such polypeptides.

The above objects highlight certain aspects of the invention. Additional objects, aspects and embodiments of the invention are found in the following detailed description of the invention.

### BRIEF DESCRIPTION OF THE DRAWINGS

FIG. 1: Map of the plasmid pCR2.1hisC2int.

### DETAILED DESCRIPTION OF THE INVENTION

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989) and the various references cited therein.

The invention provides a polynucleotide isolated from Coryneform bacteria containing a polynucleotide sequence coding for the hisC2 gene, selected from the group comprising

a) polynucleotide which is at least 70% identical to a polynucleotide coding for a polypeptide which contains the amino acid sequence of SEQ ID No. 2,

b) polynucleotide which codes for a polypeptide containing an amino acid sequence which is at least 70% identical to the amino acid sequence of SEQ ID No. 2,

c) polynucleotide which is complementary to the polynucleotides of a) or b), and

d) polynucleotide containing at least 15 consecutive nucleotides of the polynucleotide sequence of a), b) or c),

wherein the polypeptide preferably has the activity of histidinol phosphate aminotransferase.

The invention also provides the above-mentioned polynucleotide, preferably being a replicable DNA containing:

(i) the nucleotide sequence shown in SEQ ID No. 1, or

(ii) at least one sequence which corresponds to the sequence (i) within the degeneracy of the genetic code, or

(iii) at least one sequence that hybridizes with the sequences that are complementary to sequences (i) or (ii), and optionally

(iv) sense mutations in (i) that are neutral in terms of function.

The invention also provides:

a replicable DNA containing the nucleotide sequence shown in SEQ ID No. 1;

a polynucleotide that codes for a polypeptide containing the amino acid sequence shown in SEQ ID No. 2;

a vector containing parts of the polynucleotide shown in SEQ ID No. 1 that contains at least 15 consecutive nucleotides of said polynucleotide,

and Coryneform bacteria that contains the vector carrying the hisC2 gene or in which the hisC2 gene is attenuated, in particular by an insertion or deletion.

The invention also provides polynucleotides consisting substantially of a polynucleotide sequence which are obtainable by screening by means of hybridization, of a Coryneform gene library containing the complete gene having the polynucleotide sequence shown in SEQ ID No. 1, using a probe containing the sequence of said polynucleotide or a fragment thereof, and isolating the polynucleotide sequence mentioned.

Polynucleotide sequences according to the invention are suitable as hybridization probes for RNA, cDNA and DNA, in order to isolate nucleic acids or polynucleotides or full-length genes that code for histidinol phosphate aminotransferase or in order to isolate those nucleic acids or polynucleotides or genes that exhibit a high similarity with the sequence of the hisC2 gene. The hybridization probes are also suitable for incorporation in arrays, micro-arrays, or DNA chips, in order to detect and determine the corresponding polynucleotides.

The DNA of genes that code for the histidinol phosphate aminotransferase can be prepared with the polymerase chain reaction (PCR) by using the polynucleotides according to the invention as primers.

Those oligonucleotides acting as probes or primers contain at least 25, 26, 27, 28, 29 or 30, preferably at least 20, 21, 22, 23 or 24, most preferably at least 15, 16, 17, 18 or 19 consecutive nucleotides. Oligonucleotides with a length of at least 31, 32, 33, 34, 35, 36, 37, 38, 39 or 40, or at least 41, 42, 43, 44, 45, 46, 47, 48, 49 or 50 nucleotides are also suitable. Optionally, oligonucleotides with a length of at least 100, 150, 200, 250 or 300 nucleotides are also suitable.

“Isolated” means separated from its natural surroundings.

“Polynucleotide” refers generally to polyribonucleotides and polydeoxyribonucleotides. The RNA or DNA may be modified or unmodified.

Polynucleotides according to the invention include a polynucleotide shown inSEQ ID No. 1, or a fragment prepared therefrom, and also those which are at least 70% to 80%, preferably at least 81% to 85%, more preferably at least 86% to 90%, and most preferably at least 91%, 93%, 95%, 97% or 99% identical to the polynucleotide according to SEQ ID No. 1 or a fragment prepared therefrom.

“Polypeptides” are understood to be peptides or proteins that contain two or more amino acids linked via peptide bonds.

Polypeptides according to the invention include a polypeptide according to SEQ ID No. 2, particularly those with the biological activity of histidinol phosphate aminotransferase, and also those that are at least 70% to 80%, preferably at least 81% to 85%, more preferably at least 86% to 90%, and most preferably at least 91%, 93%, 95%, 97% or 99% identical to the polypeptide according to SEQ ID No. 2 and exhibit the mentioned activity.

The invention also provides a process for the production of amino acids selected from the group comprising L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine using Coryneform bacteria that, in particular, already produce amino acids and in which the nucleotide sequences coding for the hisC2 gene are attenuated, in particular switched off or expressed at a low level.

The term “attenuation” in this connection describes the reduction or exclusion of the intracellular activity of one or more enzymes (proteins) in a microorganism that are coded for by the corresponding DNA, by, for example, using a weak promotor or a gene or allele which codes for a corresponding enzyme with a low activity or by inactivating the corresponding gene or enzyme (protein), and optionally combining these measures. As a result of the attenuation, the activity or concentration of the corresponding protein is, in general, reduced to 0 to 75%, 0 to 50%, 0 to 25%, 0 to 10% or 0 to 5% of the wild-type protein activity or concentration.

Microorganisms provided by the present invention may produce L-amino acids from glucose, sucrose, lactose, fructose, maltose, molasses, starch, cellulose or from glycerol and ethanol. These microorganisms may be representatives of Coryneform bacteria, in particular of the genus Corynebacterium. Corynebacterium glutamicum of this genus garners special mention since it is well known to those skilled in the art for its ability to produce L-amino acids.

Suitable strains of the genus Corynebacterium, particularly of the species Corynebacterium glutamicum (C. glutamicum), are especially the known wild-type strains

Corynebacterium glutamicum ATCC13032

Corynebacterium acetoglutamicum ATCC15806

Corynebacterium acetoacidophilum ATCC13870

Corynebacterium melassecola ATCC17965

Corynebacterium thermoaminogenes FERM BP-1539

Brevibacterium flavum ATCC14067

Brevibacterium lactofermentum ATCC13869 and

Brevibacterium divaricatum ATCC14020

or L-amino acid-producing mutants or strains prepared therefrom.

Preferably, a bacterial strain with attenuated expression of a hisC2 that encodes a polypeptide with histidinol phosphate aminotrasferase activity will improve amino acid yields at least 1%.

The inventors have succeeded in isolating the hisC2 gene from C. glutamicum that codes for histidinol phosphate aminotransferase (EC 2.6.1.9).

To isolate the his C2 gene, or other genes, from C. glutamicum, a gene library of that microorganism is first prepared in Escherichia coli (E. coli). The preparation of gene libraries is described in generally known textbooks and manuals. For example, the textbook by Winnacker: Gene und Klone, Eine Einführung in die Gentechnologie (Verlag Chemie, Weinheim, Germany, 1990), or the manual by Sambrook et al.: Molecular Cloning, A Laboratory Manual (Cold Spring Harbor Laboratory Press, 1989). A well-known gene library is that of the E. coli K-12 strain W3110, which has been prepared by Kohara et al. (Cell 50, 495-508 (1987)) in λ-vectors. Bathe et al. (Molecular and General Genetics, 252:255-265, 1996) describe a gene library of C. glutamicum ATCC13032, which was prepared with the aid of the cosmid vector SuperCos I (Wahl et al., 1987, Proceedings of the National Academy of Sciences USA, 84:2160-2164) in E. coli K-12 strain NM554 (Raleigh et al., 1988, Nucleic Acids Research 16:1563-1575). Börmann et al. (Molecular Microbiology 6(3), 317-326 (1992)) describe a gene library of C. glutamicum ATCC13032 using the cosmid pHC79 (Hohn and Collins, 1980, Gene 11, 291-298).

It is possible to use plasmids such as pBR322 (Bolivar, 1979, Life Sciences, 25, 807-818) or pUC9 (Vieira et al., 1982, Gene, 19:259-268) to produce a gene library of C. glutamicum in E. coli. Suitable hosts are particularly E. coli strains that are restriction and recombination deficient such as the DH5αmcr strain which was described by Grant et al. (Proceedings of the National Academy of Sciences USA, 87 (1990) 4645-4649).

The long DNA fragments cloned with the aid of cosmids or other λ-vectors may then be subcloned into suitable vectors commonly used for DNA sequencing. Methods for DNA sequencing are described inter alia in Sanger et al. (Proceedings of the National Academy of Sciences of the United States of America, 74:5463-5467, 1977).

The resulting DNA sequences may then be analyzed with well-known algorithms or sequence-analysis programs such as the program by Staden (Nucleic Acids Research 14, 217-232(1986)), the program by Marck (Nucleic Acids Research 16, 1829-1836 (1988)) or the GCG program by Butler (Methods of Biochemical Analysis 39, 74-97 (1998)).

In that manner the novel DNA sequence from C. glutamicum that codes for the hisC2 gene (SEQ ID No. 1) has been obtained and forms part of the present invention. Furthermore, the amino acid sequence of the corresponding protein was derived from the available DNA sequence using the methods described above. SEQ ID No. 2 represents the amino acid sequence of the resulting hisC2 gene product.

Coding DNA sequences that are produced from SEQ ID No. 1 by degeneracy of the genetic code also form part of the invention. In the same way, DNA sequences that hybridize with SEQ ID No. 1 or parts of SEQ ID No. 1 form part of the invention. Furthermore, to a person skilled in the art, conservative amino acid exchanges, such as replacement of glycine by alanine or of aspartic acid by glutamic acid, in proteins are known as sense mutations. These mutations do not lead to a fundamental change in the activity of the protein, i.e., they are neutral in terms of function. It is also known that changes at the N and/or C terminus of a protein do not substantially impair, or may even stabilize, its function. A person skilled in the art will find relevant information inter alia in Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in well known textbooks of genetics and molecular biology. Amino acid sequences that are produced in a corresponding manner from SEQ ID No. 2 also form part of the invention. Similarly, DNA sequences that hybridize with SEQ ID No. 1 or parts of SEQ ID No. 1 form part of the invention.

Finally, DNA sequences that are prepared by the polymerase chain reaction (PCR) using primers that result from SEQ ID No. 1 form part of the invention. Such oligonucleotides typically have a length of at least 15 nucleotides.

A person skilled in the art will find instructions for identificating DNA sequences by hybridization inter alia in the manual “The DIG System Users Guide for Filter Hybridization” from Boehringer Mannheim GmbH (Mannheim, Germany, 1993) and in Liebl et al. (International Journal of Systematic Bacteriology 41: 255-260 (1991)). Hybridization takes place under stringent conditions, that is the only hybrids formed are those in which probe and target sequence, i.e. the polynucleotides treated with the probe, are at least 70% identical. It is known that the stringency of hybridization, including the washing steps is influenced or determined by varying the buffer composition, temperature, and salt concentration. For reasons explained infra, the hybridization reaction is preferably performed with relatively low stringency compared with the wash steps (Hybaid Hybridisation Guide, Hybaid Limited, Teddington, UK, 1996).

A 5× SSC buffer at a temperature of about 50° C.-68° C. can be used for the hybridization reaction. Probes may also hybridize with polynucleotides that are less than 70% identical to the sequence of the probe. Such hybrids are less stable and are removed by washing under stringent conditions. This may be achieved, for example, by lowering the salt concentration to 2× SSC and optionally then to 0.5× SSC (The DIG System User's Guide for Filter Hybridisation, Boehringer Mannheim, Mannheim, Germany, 1995), wherein the adjustments are performed at a temperature of about 50° C.-68° C. It is also possible to reduce the salt concentration to as low as 0.1× SSC. By a stepwise increase in the hybridization temperature from 50° C. to 68° C. in increments of about 1-2° C., polynucleotide fragments can be isolated that are, for example, at least 70% or at least 80% or at least 90% to 95% identical to the sequence of the probe used. Commercial kits containing further instructions for hybridization are readily obtainable on the market (e.g. DIG Easy Hyb from Roche Diagnostics GmbH, Mannheim, Germany, Catalog No. 1603558).

A person skilled in the art will find instructions for amplifying DNA sequences with the aid of the polymerase chain reaction (PCR) inter alia in the manual by Gait: Oligonucleotide Synthesis: A Practical Approach (IRL Press, Oxford, UK, 1984) and in Newton and Graham: PCR (Spektrum Akademischer Verlag, Heidelberg, Germany, 1994).

Suring work on the present invention, it was found that Coryneform bacteria produce amino acids in an improved manner after attenuation of the hisC2 gene.

To achieve attenuation, either the expression of the hisC2 gene or the catalytic properties of the enzyme protein may be reduced or excluded. Optionally, both measures may be combined.

The reduction in gene expression may be effected by performing the culturing in a suitable manner or by genetic modification (mutation) of the signal structures of gene expression. Signal structures of gene expression include, for example, repressor genes, activator genes, operators, promoters, attenuators, ribosome binding sites, the start codon, and terminators. A person skilled in the art will find information on this in patent application WO 96/15246, in Boyd and Murphy (Journal of Bacteriology 170: 5949 (1988)), in Voskuil and Chambliss (Nucleic Acids Research 26: 3548 (1998), in Jensen and Hammer (Biotechnology and Bioengineering 58: 191 (1998)), in Pátek et al. (Microbiology 142: 1297 (1996)), Vasicova et al. (Journal of Bacteriology 181: 6188 (1999)) and in known textbooks of genetics and molecular biology, such as the textbook by Knippers (“Molekulare Genetik”, 6. edition, Georg Thieme Verlag, Stuttgart, Germany, 1995) or the textbook by Winnacker (“Gene und Klone”, VCH Verlagsgesellschaft, Weinheim, Germany, 1990).

Mutations that lead to a change or reduction in the catalytic properties of enzyme proteins are known from the prior art; examples that may be mentioned are the works by Qiu and Goodman (Journal of Biological Chemistry 272: 8611-8617 (1997)), Sugimoto et al. (Bioscience Biotechnology and Biochemistry 61: 1760-1762 (1997)) and Möckel (“Die Threonindehydratase aus Corynebacterium glutamicum: Aufhebung der allosterischen Regulation und Struktur des Enzyms”, Berichte des Forschungszentrums Jülichs, Jül-2906, ISSN09442952, Jülich, Germany, 1994). Summaries found in the well-known textbooks on genetics and molecular biology such as that of Hagemann (“Allgemeine Genetik”, Gustav Fischer Verlag, Stuttgart, 1986).

These mutations may be transitions, transversions, insertions and deletions. Depending on the effect of the amino acid exchange on the enzyme activity, missense mutations or nonsense mutations are referred to. Insertions or deletions of at least one base pair (bp) in a gene lead to frame shift mutations, as a consequence incorrect amino acids are incorporated or translation is terminated prematurely. Deletions of several codons typically lead to a complete loss of enzyme activity. Instructions on producing these types of mutations are part of the prior art and may be found in well-known textbooks of genetics and molecular biology such as the textbook by Knippers (“Molekulare Genetik”, 6. edition, Georg Thieme Verlag, Stuttgart, Germany, 1995), the book by Winnacker (“Gene und Klone”, VCH Verlagsgesellschaft, Weinheim, Germany, 1990), or the book by Hagemann (“Allgemeine Genetik”, Gustav Fischer Verlag, Stuttgart, 1986).

A common method of mutating genes of C. glutamicum is the method of gene disruption and gene replacement described by Schwarzer and Pühler (Bio/Technology 9, 84-87 (1991)).

In the gene disruption method, a central part of the coding region of the gene of interest is cloned into a plasmid vector which can replicate in a host (typically E. coli), but not in C. glutamicum. Suitable vectors are, for example, pSUP301 (Simon et al., Bio/Technology 1, 784-791 (1983)), pK18mob or pK19mob (Schäfer et al., Gene 145, 69-73 (1994)), pK18mobsacB or pK19mobsacB (Jäger et al., Journal of Bacteriology 174: 5462-65 (1992)), pGEM-T (Promega corporation, Madison, Wis., USA), pCR2.1-TOPO (Shuman (1994). Journal of Biological Chemistry 269:32678-84; U.S. Pat. No. 5,487,993), pCR®Blunt (Invitrogen, Groningen, Netherlands; Bernard et al., Journal of Molecular Biology, 234: 534-541 (1993)), or pEM1 (Schrumpf et al, 1991, Journal of Bacteriology 173:4510-4516). The plasmid vector containing the central part of the coding region of the gene is then transferred in to the desired strain of C. glutamicum by conjugation or transformation. The method of conjugation is described, for example, in Schäfer et al. (Applied and Environmental Microbiology 60, 756-759 (1994)). Methods of transformation are described, for example, in Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a cross-over event, the coding region of the gene in question is disrupted by the vector sequence and two incomplete alleles are obtained, one lacking the 3′- and one lacking the 5′-end. This method was used by Fitzpatrick et al. (Applied Microbiology and Biotechnology 42, 575-580 (1994)) to exclude the recA gene in C. glutamicum.

In the gene replacement method, a mutation such as a deletion, insertion, or base replacement is produced in vitro in the gene of interest. The allele produced is in turn cloned into a vector that is not replicated in C. glutamicum and this vector is then transferred into the desired host for C. glutamicum by transformation or conjugation. After homologous recombination by means of a first cross-over event effecting integration and by means of a suitable second cross-over event effecting excision in the target gene or in the target sequence, incorporation of the mutation or the allele is achieved. This method was used by Peters-Wendisch et al. (Microbiology 144, 915-927 (1998)) to exclude the pyc gene in C. glutamicum by deletion.

A deletion, insertion, or a base replacement can be incorporated in the hisC2 gene in this way.

In addition, it may be advantageous for the production of L-amino acids, in addition to attenuating the hisC2 gene, to amplify, in particular overexpress, one or more enzymes involved in glycolysis, anaplerotic reaction, the citric acid cycle, the pentose phosphate cycle, amino acid export, and, optionally, regulatory proteins.

The term “enhancement” in this connection describes the increase in intracellular activity of one or more enzymes (proteins) in a microorganism that are coded for by the corresponding DNA by, for example, increasing the copy number of the gene or genes, using a strong promotor, or using a gene or allele that codes for a corresponding enzyme (protein) with a high activity and optionally combining these methods.

As a result of enhancement, in particular overexpression, the activity or concentration of the corresponding protein is increased, in general, preferably ranging from at least 10%, 25%, 50%, 75%, 100%, 150%, 200%, 300%, 400% or 500%, up to 1000% or 2000% of the wild-type protein activity or concentration present in the microorganism.

Thus, for example, for the production of L-amino acids, one or more of the genes chosen from the group

the dapA gene coding for dihydrodipicolinate synthase (EP-B 0 197 335),

the gap gene coding for glyceraldehyde-3-phosphate dehydrogenase (Eikmanns (1992), Journal of Bacteriology 174:6076-6086),

the tpi gene coding for triosephosphate isomerase (Eikmanns (1992), Journal of Bacteriology 174:6076-6086),

the pgk gene coding for 3-phosphoglycerate kinase (Eikmanns (1992), Journal of Bacteriology 174:6076-6086),

the zwf gene coding for glucose-6-phosphate dehydrogenase (JP-A-09224661),

the pyc gene coding for pyruvate carboxylase (DE-A-198 31 609),

the mqo gene coding for malate quinone oxidoreductase (Molenaar et al., European Journal of Biochemistry 254, 395-403 (1998)),

the lysC gene coding for a feedback resistant aspartate kinase (EP-B-0387527; EP-A-0699759; WO 00/63388),

the lysE gene coding for lysine export (DE-A-195 48 222),

the hom gene coding for homoserin dehydrogenase EP-A 0131171),

the ilvA gene coding for threonine dehydratase (Möckel et al., Journal of Bacteriology (1992) 8065-8072)) or the ilvA(Fbr) allele coding for a feedback resistant

threonine dehydratase (Möckel et al., (1994) Molecular Microbiology 13: 833-842),

the ilvBN gene coding for acetohydroxy acid synthase (EP-B 0356739),

the ilvD gene coding for dihydroxy acid dehydratase (Sahm and Eggeling (1999) Applied and Environmental Microbiology 65: 1973-1979),

the zwal gene coding for the Zwa1 protein (DE: 19959328.0, DSM 13115)

may be enhanced, in particular overexpressed, simultaneously.

It may also be advantageous for the production of amino acids, in addition to attenuating the hisC2 gene, to simultaneously attenuate one or more of the genes chosen from the group

the pck gene coding for phosphoenolpyruvate carboxykinase (DE 199 50 409.1, DSM 13047),

the pgi gene coding for glucose-6-phosphate isomerase (U.S. Ser. No. 09/396,478, DSM 12969),

the poxB gene coding for pyruvate oxidase (DE:1995 1975.7, DSM 13114),

the zwa2 gene coding for the Zwa2 protein (DE: 19959327.2, DSM 13113).

Furthermore, it may be advantageous for the production of amino acids, in addition to attenuating the hisC2 gene, to eliminate unwanted side reactions (Nakayama: “Breeding of Amino Acid Producing Microorganisms”, in: Overproduction of Microbial Products, Krumphanzl, Sikyta, Vanek (eds.), Academic Press, London, UK, 1982).

Microorganisms prepared according to the invention also, for purposes of producing L-amino acids, may be cultured continuously or batchwise in a batch process, fed-batch process, or repeated fed-batch process. A summary of well-known culture methods is described in the textbook by Chmiel (Bioprozesstechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

A suitable culture medium must be used to meet the requirements of the particular strain. Descriptions of culture media for various microorganisms are contained in the manual “Manual of Methods for General Bacteriology” of the American Society for Bacteriology (Washington D.C., USA, 1981).

The carbon sources may be sugars and carbohydrates (e.g., glucose, sucrose, lactose, fructose, maltose, molasses, starch and cellulose), oils and fats (e.g., soyabean oil, sunflower oil, groundnut oil and coconut oil), fatty acids (e.g., palmitic acid, stearic acid and linoleic acid), alcohols (e.g. glycerol and ethanol), and organic acids (e.g., acetic acid). These substances may be used individually or as a mixture.

The nitrogen sources may be organic nitrogen-containing compounds (e.g., peptones, yeast extract, meat extract, malt extract, corn steep liquor, soyabean flour and urea) or inorganic compounds (e.g., ammonium sulfate, ammonium chloride, ammonium phosphate, ammonium carbonate and ammonium nitrate). The sources of nitrogen may be used individually or as a mixture.

The phosphorus sources may be phosphoric acid, potassium dihydrogen phosphate or dipotassium hydrogen phosphate (or the corresponding sodium-containing salts).

Furthermore, the culture medium must contain salts of metals (e.g., magnesium sulfate or iron sulfate) that are required for growth. Finally, essential growth-promoting substances, such as amino acids and vitamins, may be used in addition to the above-mentioned substances. Moreover, suitable precursors may be added to the culture medium. The starting substances mentioned may be added to the culture in the form of a single batch or may be fed in a suitable manner during fermentation.

Regulation of the pH of the culture may be achieved by addition of basic compounds (e.g., sodium hydroxide, potassium hydroxide, ammonia or ammonia solution) or acid compounds (e.g., phosphoric acid or sulfuric acid) in a suitable manner. Fatty acid polyglycol esters may be used to control the development of foam. In order to maintain the stability of plasmids, suitable substances having a selective action, such as antibiotics, may be added to the medium. In order to maintain aerobic conditions, oxygen or oxygen-containing gas mixtures, such as air, may be introduced into the culture. The temperature of the culture is normally 20° C. to 45° C. and is preferably from 25° C. to 40° C. Fermentation is continued until the maximum of the desired product has been formed. This objective is normally achieved within 10 hours to 160 hours.

Methods for the determination L-amino acids are known from the prior art. The analysis may be performed, for example, by anion exchange chromatography followed by ninhydrin derivation as described by Spackman et al. (Analytical Chemistry, 30, (1958), 1190) or it may be performed by reversed phase HPLC as described by Lindroth et al. (Analytical Chemistry (1979) 51: 1167-1174).

The process according to the invention is used for the production of amino acids by fermentation.

The isolation of plasmid DNA from Escherichia coli and all techniques of restriction, Klenow and alkaline phosphatase treatment were performed as described in Sambrook et al. (Molecular Cloning. A Laboratory Manual (1989) Cold Spring Harbor Laboratory Press, Cold Spring Harbor, N.Y., USA). Methods for transformation of Escherichia coil and the composition of the usual nutrient media, such as LB or TY medium, are also described in this handbook.

The following microorganism was deposited on Dec. 1, 2001 at the German Collection for Microorganisms and Cell Cultures (DSMZ, Braunschweig, Germany) in accordance with the Budapest Agreement:

Escherichia coli Top10/pCR2.1hisC2int as DSM 13984.

Having generally described this invention, a further understanding can be obtained by reference to certain specific examples, which are provided herein for purposes of illustration only, and are not intended to be limiting unless otherwise specified.

### EXAMPLES

### Example 1

Production of a Genomic Cosmid Gene Library from C. glutamicum ATCC 13032

Chromosomal DNA from C. glutamicum ATCC 13032 was isolated as described in Tauch et al. (1995, Plasmid 33:168-179), and partially cleaved with the restriction enzyme Sau3AI (Amersham Pharmacia, Freiburg, Germany, product description Sau3AI, code no. 27-0913-02). The DNA fragments were dephosphorylated with shrimp alkaline phosphatase (Roche Molecular Biochemicals, Mannheim, Germany, product description SAP, code no. 1758250). The DNA of the cosmid vector SuperCos1 (Wahl et al. (1987) Proceedings of the National Academy of Sciences USA 84:2160-2164), purchased from the company Stratagene (La Jolla, USA, product description SuperCos1 Cosmid Vector Kit, code no. 251301) was cleaved with the restriction enzyme XbaI (Amersham Pharmacia, Freiburg, Germany, product description XbaI, code no. 27-0948-02) and also dephosphorylated with shrimp alkaline phosphatase.

The cosmid DNA was then cleaved with the restriction enzyme BamHI (Amersham Pharmacia, Freiburg, Germany, product description BamHI, code no. 27-0868-04). The cosmid DNA so treated was mixed with the treated ATCC 13032-DNA and the mixture was additionally treated with T4-DNA-ligase (Amersham Pharmacia, Freiburg, Germany, product description T4-DNA-Ligase, code no.27-0870-04). The ligation mixture was then packaged into phages using Gigapack II XL Packing Extracts (Stratagene, La Jolla, USA, product description Gigapack II XL Packing Extract, code no. 200217).

For infection of the E. coli strain NM554 (Raleigh et al. 1988, Nucleic Acid Research 16:1563-1575) the cells were taken up in 10 mM MgSO₄ and mixed with an aliquot of the phage suspension. Infection and titering of the cosmid library were performed as described in Sambrook et al. (1989, Molecular Cloning: A Laboratory Manual, Cold Spring Harbor), the cells being plated on LB agar (Lennox, 1955, Virology, 1:190) with 100 μg/ml ampicillin. After incubation overnight at 37° C., recombinant individual clones were selected.

### Example 2

Isolation and Sequencing of the hisC2 Gene

The cosmid DNA from an individual colony was isolated with the Qiaprep Spin Miniprep Kit product no. 27106, Qiagen, Hilden, Germany) in accordance with the manufacturer's instructions and partially cleaved with the restriction enzyme Sau3AI (Amersham Pharmacia, Freiburg, Germany, product description Sau3AI, product no. 27-0913-02). The DNA fragments were dephosphorylated with shrimp alkaline phosphatase (Roche Molecular Biochemicals, Mannheim, Germany, product description SAP, product no. 1758250). After separation by gel electrophoresis, isolation of the cosmid fragments in the size region from 1500 to 2000 bp was carried out with the QiaExII Gel Extraction Kit (product no. 20021, Qiagen, Hilden, Germany).

The DNA of the sequencing vector pZero-1 purchased from Invitrogen (Groningen, the Netherlands, product description Zero Background Cloning Kit, product no. K2500-01) was cleaved with the restriction enzyme BamHI (Amersham Pharmacia, Freiburg, Germany, product description BamHI, product no. 27-0868-04). Ligation of the cosmid fragments into the sequencing vector pZero-1 was carried out as described by Sambrook et al. (1989, Molecular Cloning: A Laboratory Manual, Cold Spring Harbor), the DNA mixture being incubated overnight with T4-ligase (Pharmacia Biotech, Freiburg, Germany). This ligation mixture was then electroporated into the E. coli strain DH5αMCR (Grant, 1990, Proceedings of the National Academy of Sciences U.S.A., 87:4645-4649)(Tauch et al. 1994, FEMS Microbiol Letters, 123:343-7) and plated on LB agar (Lennox, 1955, Virology, 1:190) with 50 μg/ml Zeocin.

Plasmid preparation of the recombinant clones was performed with the Biorobot 9600 (product no. 900200, Qiagen, Hilden, Germany). DNA sequencing was performed by the dideoxy-chain termination method according to Sanger et al. (1977, Proceedings of the National Academy of Sciences U.S.A., 74:5463-5467) with modifications by Zimmermann et al. (1990, Nucleic Acids Research, 18:1067). The “RR dRhodamin Terminator Cycle Sequencing Kit” from PE Applied Biosystems (product no. 403044, Weiterstadt, Germany) was used. Gel electrophoretic separation and analysis of the sequencing reaction was performed in a “Rotiphoresis NF acrylamide/bisacrylamide” gel (29:1) (product no. A124.1, Roth, Karlsruhe, Germany) with the “ABI Prism 377” sequencing device from PE Applied Biosystems (Weiterstadt, Germany).

The raw sequencing data obtained were then processed using the Staden program package (1986, Nucleic Acids Research, 14:217-231) version 97-0. The individual sequences of the pZero 1 derivatives were assembled to give a coherent contig. Computer aided coding region analyses were prepared with the program XNIP (Staden, 1986, Nucleic Acids Research, 14:217-231). Further analyses were performed with the “BLAST search programs” (Altschul et al., 1997, Nucleic Acids Research, 25:3389-3402), against the non-redundant data base of the “National Center for Biotechnology Information” (NCBI, Bethesda, Md., USA).

The resulting nucleotide sequence obtained is shown in SEQ ID No. 1. Analysis of the nucleotide sequence revealed an open reading frame of 1026 base pairs, which was designated the hisC2 gene. The hisC2 gene codes for a polypeptide of 341 amino acids.

### Example 3

Preparation of an Integration Vector for Integration Mutagenesis of the hisC2 Gene

From the C. glutamicum strain ATCC 13032, chromosomal DNA was isolated by the method of Eikmanns et al. (Microbiology 140: 1817-1828 (1994)). On the basis of the sequence of the hisC2 gene known for C. glutamicum from Example 2, the following oligonucleotides were selected for the polymerase chain reaction:

hisC2-int1 (SEQ ID No. 3):

5′ GCA GCT TTG AGG CTT ATC C 3′

hisC2-int2 (SEQ ID No. 4):

5′ AGA ATT CAA ACT CGC AAG C 3′

The primers shown were synthesized by MWG Biotech (Ebersberg, Germany) and the PCR reaction was carried out according to the standard PCR method of Innis et al. (PCR protocols. A guide to methods and applications, 1990, Academic Press) with Taq-polymerase from Boehringer Mannheim (Germany, product description Taq DNA Polymerase, product no. 1 146 165). With the aid of the polymerase chain reaction, a 467 bp internal fragment of the hisC2 gene was isolated. The product thus amplified was tested electrophoretically in a 0.8% agarose gel.

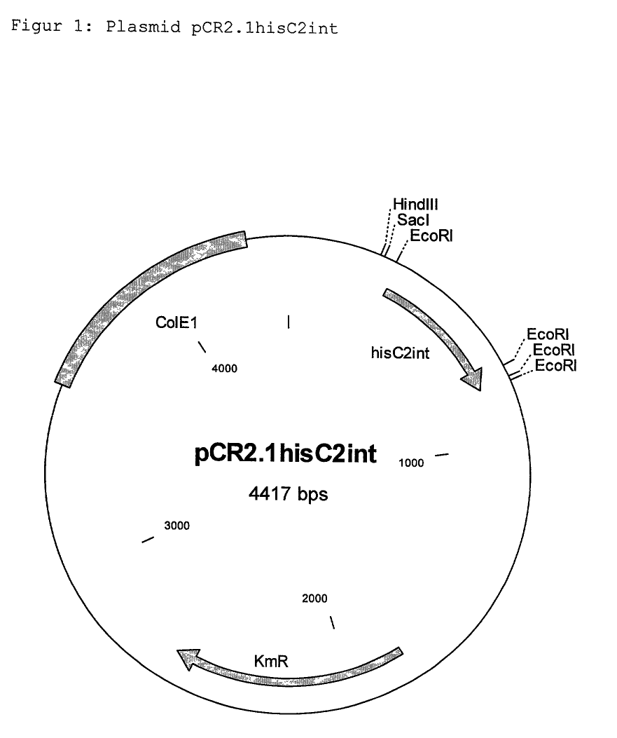

The amplified DNA fragment was ligated with the TOPO TA Cloning Kit from Invitrogen Corporation (Carlsbad, Calif., USA; catalogue number K4500-01) into the vector pCR2.1-TOPO (Mead at al. (1991) Bio/Technology 9:657-663) and subsequently transformed into the E. coli Stamm TOP10 (Hanahan, In: DNA Cloning. A Practical Approach. Vol. I, IRL-Press, Oxford, Washington D.C., USA, 1985). Plasmid-carrying cells were selected by plating the transformation mix onto LB agar (Sambrook et al., Molecular cloning: a laboratory manual. 2^{nd} Ed. Cold Spring Harbor Laboratory Press, Cold Spring Harbor, N.Y., 1989), which had been supplemented with 50 mg/l kanamyin. Plasmid DNA was isolated from a transformant using the QIAprep Spin Miniprep Kit from Qiagen and analysed by restriction with the restriction enzyme EcoRI followed by agarose gel electrophoresis (0.8%). The plasmid was named pCR2.1hisC2int and is shown in FIG. 1.

### Example 4

Integration Mutagenesis of the hisC2 Gene in the Strain DSM 5715

Corynebacterium glutamicum DSM 5715 was transformed with the vector pCR2.1hisC2int from Example 3 according to the method of Tauch et al.(FEMS Microbiological Letters, 123:343-347 (1994)). The strain DSM 5715 is an AEC resistant lysine producer. The vector pCR2.1hisC2int is unable to replicate of its own accord in DSM5715 and only remains in the cell if it has integrated in the chromosome of DSM 5715. Clones containing pCR2.1hisC2int chromosomal integrates were selected by plating the electroporation mix onto LB agar (Sambrook et al., Molecular cloning: a laboratory manual. 2^{nd} Ed. Cold Spring Harbor Laboratory Press, Cold Spring Harbor, N.Y.), which had been supplemented with 15 mg/l kanamyin.

For the detection of integration, the hisC2int fragment was labeled with the Dig hybridization kit from Boehringer using the method “The DIG System Users Guide for Filter Hybridization” of Boehringer Mannheim GmbH (Mannheim, Germany, 1993). Chromosomal DNA of a potential integrant was isolated by the method of Eikmanns et al. (Microbiology 140: 1817-1828 (1994)) and cut in each case with the restriction enzymes SacI, EcoRI and HindIII. The resulting fragments were separated using agarose gel electrophoresis and hybridized with the Dig hybridization kit from Boehringer at 68° C. The plasmid pCR2.1hisC2int from Example 3 was found in the chromosome of DSM5715 within the chromosomal hisC2 gene. The strain was named DSM5715:pCR2.1hisC2int.

### Example 5

Preparation of L-lysine

The C. glutamicum strain DSM5715:pCR2.1hisC2int obtained in Example 4 was cultured in a nutrient medium suitable for the production of L-lysine by fermentation, and the L-lysine content in the culture supernatant was determined.

To that end, the strain was first incubated on an agar plate with the corresponding antibiotic (brain-heart agar with kanamyin (25 mg/l) for 24 hours at 33° C. A pre-culture was inoculated (10 ml medium in a 100 ml Erlenmeyer flask). The complete CgIII medium was used as the medium for the pre-culture starting from this agar plate culture.

CgIII Medium

| | |
|---|---|
| NaCl | 2.5 g/l |
| Bacto-peptone | 10 g/l |
| Bacto-yeast-extract | 10 g/l |
| Glucose (autoclaved separately) | 2% (w/v) |
| The pH was adjusted to 7.4 | |

Kanamyin (25 mg/l) was added to the pre-culture medium. The pre-culture was incubated for 16 hours at 33° C. at 240 rpm on the shaker. A main culture was inoculated from this pre-culture so that the initial OD (660 nm) of the main culture was 0.1 OD. MM medium was used for the main culture.

Medium MM

| | | |
|---|---|---|
| CSL (Corn Steep Liquor) | 5 | g/l |
| MOPS | 20 | g/l |
| Glucose (autoclaved separately) | 50 | g/l |
| Salts: | | |
| (NH₄)₂SO₄ | 25 | g/l |
| KH₂PO₄ | 0.1 | g/l |
| MgSO₄ * 7H₂O | 1.0 | g/l |
| CaCl₂ * 2H₂O | 10 | mg/l |
| FeSO₄ * 7H₂O | 10 | mg/l |
| MnSO₄ * H₂O | 5.0 | mg/l |
| Biotin (filter-sterilised) | 0.3 | mg/l |
| Thiamine * HCl (filter-sterilised) | 0.2 | mg/l |
| Leucine (filter-sterilised) | 0.1 | g/l |
| CaCO₃ | 25 | g/l |

CSL, MOPS and the salt solution were adjusted to pH 7 with aqueous ammonia and autoclaved. The sterile substrate and vitamin solutions were then added, as well as the dry, autoclaved CaCO₃.

Cell-growth was performed in a 10 ml volume in a 100 ml Erlenmeyer flask with baffles. Kanamyin (25 mg/l) was added. Culturing was carried out at 33° C. and at 80% atmospheric humidity.

After 72 hours, the OD was determined at a measurement wavelength of 660 nm with the Biomek 1000 (Beckmann Instruments GmbH, Munich). The amount of L-lysine formed was determined with an amino acid analyzer from Eppendorf-BioTronik (Hamburg, Germany) by ion exchange chromatography and post-column derivation with ninhydrin detection.

The result of the test is shown in Table 1.

TABLE 1

| Strain | OD(660 nm) | Lysine-HCl (g/l) |
|---|---|---|
| DSM5715 | 8.2 | 13.74 |
| DSM5715::pCR2.1hisC2int | 8.7 | 14.75 |

Obviously, numerous modifications and variations on the present invention are possible in light of the above teachings. It is therefore to be understood that within the scope of the appended claims, the invention may be practiced otherwise than as specifically described herein.

What is claimed is:

1. An isolated polynucleotide, which encodes a protein comprising the amino acid sequence of SEQ ID NO:2.

2. The isolated polynucleotide of claim 1, wherein said protein has histidinol phosphate aminotransferase activity.

3. A vector comprising the isolated polynucleotide of claim 1.

4. A host cell comprising the isolated polynucleotide of claim 1.

5. The host cell of claim 4, which is a Coryneform bacterium.

6. The host cell of claim 4, wherein said host cell is selected from the group consisting of Coryneform glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

7. A method for detecting a nucleic acid with at least 70% homology to nucleotide of claim 1, comprising contacting a nucleic acid sample with a probe or primer comprising at least 15 consecutive nucleotides of the nucleotide sequence of claim 1, or at least 15 consecutive nucleotides of the complement thereof.

8. A method for producing a nucleic acid with at least 70% homology to nucleotide of claim 1, comprising contacting a nucleic acid sample with a primer comprising at least 15 consecutive nucleotides of the nucleotide sequence of claim 1, or at least 15 consecutive nucleotides of the complement thereof.

9. A process for screening for polynucleotides, which encode a protein having histidinol phosphate aminotransferase activity comprising
a) hybridizing the isolated polynucleotide of claim 1 to the polynucleotide to be screened;
b) expressing the polynucleotide to produce a protein; and
c) detecting the presence or absence of histidinol phosphate aminotransferase activity in said protein.

10. A method for making histidinol phosphate aminotransferase protein, comprising
a) culturing the host cell of claim 4 for a duration of time under conditions suitable for expression of histidinol phosphate aminotransferase protein; and
b) collecting the histidinol phosphate aminotransferase protein.

11. An isolated polynucleotide, which comprises SEQ ID NO:1.

12. An isolated polynucleotide, which is complimentary to the polynucleotide of claim 11.

13. An isolated polynucleotide, which is at least 70% identical to the polynucleotide of claim 11.

14. An isolated polynucleotide, which is at least 80% identical to the polynucleotide of claim 11.

15. An isolated polynucleotide, which is at least 90% identical to the polynucleotide of claim 11.

16. An isolated polynucleotide, which comprises at least 15 consecutive nucleotides of the polynucleotide of claim 11.

17. An isolated polynucleotide, which hybridizes under stringent conditions to the polynucleotide of claim 11; wherein said stringent conditions comprise washing in 5× SSC at a temperature from 50 to 68° C.

18. The isolated polynucleotide of claim 11, which encodes a protein having histidinol phosphate aminotransferase activity.

19. A vector comprising the isolated polynucleotide of claim 11.

20. A host cell comprising the isolated polynucleotide of claim 11.

21. The host cell of claim 20, which is a Coryneform bacterium.

22. The host cell of claim 20, wherein said host cell is selected from the group consisting of Coryneform glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

23. A process for screening for polynucleotides, which encode a protein having histidinol phosphate aminotransferase activity comprising
a) hybridizing the isolated polynucleotide of claim 11 to the polynucleotide to be screened;
b) expressing the polynucleotide to produce a protein; and
c) detecting the presence or absence of histidinol phosphate aminotransferase activity in said protein.

24. A method for detecting a nucleic acid with at least 70% homology to nucleotide of claim 11, comprising contacting a nucleic acid sample with a probe or primer comprising at least 15 consecutive nucleotides of the nucleotide sequence of claim 11, or at least 15 consecutive nucleotides of the complement thereof.

25. A method for producing a nucleic acid with at least 70% homology to nucleotide of claim 11, comprising contacting a nucleic acid sample with a primer comprising at least 15 consecutive nucleotides of the nucleotide sequence of claim 11, or at least 15 consecutive nucleotides of the complement thereof.

26. A method for making histidinol phosphate aminotransferase protein, comprising
a) culturing the host cell of claim 20 for a duration of time under conditions suitable for expression of histidinol phosphate aminotransferase protein; and
b) collecting the histidinol phosphate aminotransferase protein.

27. A Coryneform bacterium, which comprises attenuated expression of the hisC2 gene.

28. The Coryneform bacterium of claim 27, wherein said hisC2 gene comprises the polynucleotide sequence of SEQ ID NO:1.

29. Escherichia coli DSM 13984.

30. A process for producing L-amino acids comprising culturing a bacterial cell in a medium suitable for producing L-amino acids, wherein said bacterial cell comprises attenuated expression of the hisC2 gene.

31. The process of claim 30, wherein said bacterial cell is a Coryneform bacterium or Brevibacterim.

32. The process of claim 31, wherein said bacterial cell is selected from the group consisting of Coryneform glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

33. The process of claim 30, wherein said hisC2 gene comprises the polynucleotide sequence of SEQ ID NO: 1.

34. The process of claim 30, wherein said L-amino acid is L-lysine.

35. The process of claim 30, wherein said bacteria further comprises at least one gene whose expression is enhanced, wherein said gene is selected from the group consisting of dapA, gap, tpi, pgk; zwf pyc, mqo, lysC, lysE, hom, ilvA, ilvA(Fbr), ilvBN, ilvD, and zwa1.

36. The process of claim 30, wherein said bacteria further comprises at least one gene whose expression is attenuated, wherein said gene is selected from the group consisting of pck, pgi, poxB, and zwa2.

37. An isolated polypeptide comprising the amino acid sequence of SEQ ID NO:2.


SEQUENCE LISTING (4 sequences)

SEQ ID NO: 1 - DNA, 1480 bp, Corynebacterium glutamicum
  CDS (241)..(1263)
tcgcttgaccgaaatcaagctagagcggagacgagaggatttgaacctccggtcccccgttaagaggacaactcattagcagtgagtcccattcggccgctctggcacgtctccttagttcgctcgggctggccgatagaaccgatatgttacagtaccccctcttgctaaatttcagcaaacctgcaggtaaatgacttaatcggatgcgtgtccataggggctagtactgtgtaaatcatgattagagcagatttggcaactatccctacttatgtccctggccgtcgtcttgttgatgctacgaagttatctagtaatgaagttagtttttcccctctcccggcagcagttgatgcggtgacggaggctacttggggggctaatcggtacccggatatgggtgcggttgagctccgtgaggctcttgcagagcatttagaggttgagtttgaccaggtcacggtaggttgcggctcgtctgcgctgtgtcaacagctggttcaggcaacgtgcgctcagggcgatgaggtcatttttccatggcgcagctttgaggcttatccaattttcgcgcaggtcgcgggcgccactcctgttgccattccgctgactgctgatcagaatcatgatcttgatgcgatggcagccgcgatcactgataagacccgcctcattttcatctgcaaccccaacaatccttcgggcaccaccatcacccaggcgcagtttgataatttcatggaaaaggttccaaacgatgtcgttgttgggctggatgaggcttattttgagttcaaccgcgcggacgacaccccagttgccactgaggaaatccaccgccacgacaacgtgattggtttgcgcacgttctccaaggcgtatggcctggcgggcttgcgtgttggttacgccttcggaaacgcagagatcatcgcagcgatgaataaggtggctattcctttcgcggtgaattcagcagctcaggcggcagcgcttgcgagtttgaattctgccgatgagttgatggaacgggtggaggaaaccgtcgaaaagcgtgatgctgtggtgtcagcgcttggtgctgcgccgacgcaggccaatttcgtctggctgccgggcgagggcgccgctgagttggcggctaaattggccgagcacggcatcgtgattcgcgcgttccccgagggtgcgcgcatttcggtgaccaacgccgaggaaactgacaagctgctgcgcgcgtgggaggccatcaatgctgggtagtctttggcgttttgcggtgcgcaccgcagcaggcgcggtggcgttgtgggtggttattaagcttatcgacggcatctccctgagttttcccaccacacctctctatcaggacggtcagcacgacaatctgctgacattcctggcggtggcagcaatcattgtcgtgttgaatgccacggtgaaacccgtcttgaagctgcttggtttgccgttg

SEQ ID NO: 2 - protein, 341 aa, Corynebacterium glutamicum
MIRADLATIPTYVPGRRLVDATKLSSNEVSFSPLPAAVDAVTEATWGANRYPDMGAVELREALAEHLEVEFDQVTVGCGSSALCQQLVQATCAQGDEVIFPWRSFEAYPIFAQVAGATPVAIPLTADQNHDLDAMAAAITDKTRLIFICNPNNPSGTTITQAQFDNFMEKVPNDVVVGLDEAYFEFNRADDTPVATEEIHRHDNVIGLRTFSKAYGLAGLRVGYAFGNAEIIAAMNKVAIPFAVNSAAQAAALASLNSADELMERVEETVEKRDAVVSALGAAPTQANFVWLPGEGAAELAAKLAEHGIVIRAFPEGARISVTNAEETDKLLRAWEAINAG

SEQ ID NO: 3 - DNA, 19 bp, Artificial Sequence
  Synthetic DNA
gcagctttgaggcttatcc

SEQ ID NO: 4 - DNA, 19 bp, Artificial Sequence
  Synthetic DNA
agaattcaaactcgcaagc